(19) United States
(12) Patent Application Publication
(10) Pub. No.: US 2020/0281991 A1
(43) Pub. Date: Sep. 10, 2020

(54) METHODS AND COMPOSITIONS FOR TREATING DISORDERS RELATED TO A GUT DYSBIOSIS

(71) Applicant: Crestovo Holdings LLC, Somerville, MA (US)

(72) Inventors: Ylaine GERARDIN, Cambridge, MA (US); Sonia TIMBERLAKE, Brookline, MA (US); Michael SILVERSTEIN, Cambridge, MA (US)

(73) Assignee: Crestovo Holdings LLC, Somerville, MA (US)

(21) Appl. No.: 16/813,100

(22) Filed: Mar. 9, 2020

Related U.S. Application Data
(60) Provisional application No. 62/815,284, filed on Mar. 7, 2019.

(51) Int. Cl. A61K 35/74; G16H 50/30; C12Q 1/689

(52) U.S. Cl. CPC A61K 35/74; C12Q 2600/118; C12Q 1/689; G16H 50/30

(57) ABSTRACT

This application provides methods for determining or monitoring the pharmacokinetics (PK) and stable engraftment of live microbial therapeutics, in a subject, through a machine learning model. Disclosed herein is the monitoring and treating of a disorder related to a gut dysbiosis or inflammatory bowel disease in a subject.

This application claims priority to U.S. Provisional Application No. 62/815,284, filed Mar. 7, 2019, which is incorporated by reference in its entirety.

### BACKGROUND

Transplantation of healthy donor stool into patients using fecal microbiota transplantation (FMT) has shown promise in a growing number of indications, from infectious disease, to chronic inflammatory conditions, to neurological disorders. However, much remains to be learned about the mechanisms of action through which FMT works to treat disease.

Traditional drug development requires first understanding the pharmacokinetics (PK) of drug delivery, but there is no consensus on how to define or measure PK for live microbial therapeutics, where parts of the “drug” can grow inside the patients to many times the number of dosed cells, and stably engraft to become part of the patient's microbial community (FIG. 1A and FIG. 1B).

### SUMMARY

The present disclosure provides for, and includes, a method for treating a disorder related to a gut dysbiosis by increasing an abundance of a bacterial strain in an intestine of a subject, the method comprising: administering a first dose of a pharmaceutical composition comprising a preparation of uncultured fecal bacteria to the subject, wherein the preparation of uncultured fecal bacteria comprises fecal bacteria from a stool of a healthy human donor, wherein the fecal bacteria comprise the bacterial strain; and administering a second dose of the pharmaceutical composition to the subject based on a negative engraftment status of the bacterial strain in the intestine of the subject following administration of the first dose of the pharmaceutical composition; wherein the negative engraftment status is determined by processing via a machine learning model a plurality of DNA sequence-based engraftment metrics, wherein each of the plurality of DNA sequence-based engraftment metrics is representative of an attribute of at least two DNA sequences, wherein the DNA sequences are selected from the group consisting of: (i) a DNA sequence of the fecal bacteria from the stool of the healthy donor; (ii) a DNA sequence of a fecal microbiota from a stool of the subject collected prior to administering the first dose of the pharmaceutical composition; and (iii) a DNA sequence of a fecal microbiota from a stool of the subject collected after administering the first dose of the pharmaceutical composition and before administering the second dose of the pharmaceutical composition.

The present disclosure provides for, and includes, a method for treating a subject having inflammatory bowel disease, the method comprising: administering a pharmaceutical composition comprising a preparation of uncultured fecal bacteria to the subject, wherein the preparation of uncultured fecal bacteria comprises fecal bacteria from a stool of a healthy human donor, wherein the fecal bacteria comprise a bacterial strain; detecting a negative engraftment status of the bacterial strain in the intestine of the subject after administering the pharmaceutical composition, wherein the negative engraftment status is determined by processing via a machine learning model a plurality of DNA sequence-based engraftment metrics, wherein each of the plurality of DNA sequence-based engraftment metrics is representative of an attribute of at least two DNA sequences, wherein the DNA sequences are selected from the group consisting of: (i) a DNA sequence of the fecal bacteria from the stool of the healthy donor; (ii) a DNA sequence of a fecal microbiota from a stool of the subject collected prior to administering the first dose of the pharmaceutical composition; and (iii) a DNA sequence of a fecal microbiota from a stool of the subject collected after administering a first dose of the pharmaceutical composition and before administering a second dose of the pharmaceutical composition; and administering the bacterial strain to the subject based on detecting the negative engraftment status.

### BRIEF DESCRIPTION OF THE DRAWINGS

FIG. 1A shows a pharmacokinetics curve associated with the delivery of a conventional drug to a subject.

FIG. 1B shows a pharmacokinetics curve of a microbial therapeutic.

FIG. 2 illustrates a comparison of microbiota compositions in the intestines of different patients after receiving FMT using uncultured fecal bacteria derived from a single donor.

FIG. 3A shows use of marker gene abundances to track engraftment dynamics after FMT.

FIG. 3B shows use of whole genome data to track engraftment dynamics after FMT.

FIG. 3C shows the presence and absence of gene families in healthy donors and patients before and after FMT (pre-FMT and post-FMT, respectively).

FIG. 4A depicts a method to identify engraftment of a bacterial strain after transplantation of uncultured fecal bacteria from a donor into a patient recipient.

FIG. 4B shows the ability of the model to correctly detect the engraftment status of strains known to be present or absent in the intestine of a subject.

FIG. 4C shows output scores from the model for two representative strains, one of which is predicted to not have engrafted in a recipient after FMT (left), and one which is predicted to have engrafted (right).

FIG. 5A shows that the engraftment of strains from a donor microbiota following FMT varies according to bacterial species.

FIG. 5B shows engraftment stability tracked across longitudinal samples.

FIG. 5C demonstrates a correlation between engraftment and clinical response of the FMT recipient.

FIG. 6 is a block diagram of an example computer device for implementing the machine learning algorithm for predictions of engraftment.

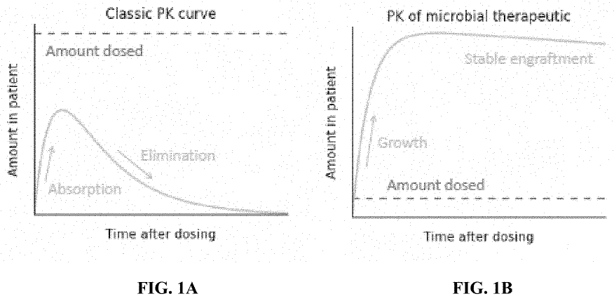

### DETAILED DESCRIPTION

Unless defined otherwise herein, terms are to be understood according to conventional usage by those of ordinary skill in the relevant art.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as percentages, density, volume and the like, is meant to encompass variations of 20%, 10%, 5%, 1%, 0.5%, or even 0.1% of the specified amount.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, a “microbiota” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. Typically a pharmaceutical composition described herein is prepared by incorporating such a fecal microbiota into the composition without culturing the fecal microbiota after its purification from a stool. Herein “uncultured fecal bacteria” or a “preparation of uncultured fecal bacteria” refer to a preparation comprising multiple non-pathogenic viable bacterial strains that have been harvested, extracted or purified from one or more stool samples, without culturing the strains (e.g. in culturing medium). Such a preparation of uncultured fecal bacteria can also be referred to as a collection of uncultured fecal bacteria or a population of uncultured fecal bacteria.

In some aspects, a preparation of uncultured fecal bacteria comprises non-selected fecal bacteria. Herein “non-selected fecal bacteria” refers to a collection of viable fecal bacterial strains (e.g., present in a fecal microbiota) extracted from one or more stool samples without subjecting the extracted bacteria to environmental conditions that intentionally select for a particular type, state or taxonomic category of bacteria (e.g., by deliberate removal of certain strains of bacteria, treatment of the bacteria with an agent such as ethanol or chloroform, or culturing). Such non-selected fecal bacteria can comprise bacterial strains in proportional content to corresponding bacterial strains in a fecal or intestinal microbiota of a normal healthy human. Steps taken to non-selectively extract fecal bacteria from a stool sample can include, for example, homogenization and filtering of the stool sample to separate the fecal bacterial strains from non-cellular stool material such as fiber and rough particulate matter, as well as, for example, eukaryotic host cells and viruses. Herein typically a non-selected fecal bacterial preparation can be prepared in either aerobic or anaerobic conditions, or a combination thereof. In certain aspects, a preparation of non-selected fecal bacteria comprises all or substantially all of the bacteria of a fecal microbiota of a stool sample. In certain aspects, a preparation of non-selected fecal bacteria comprises all or substantially all of the strains of a fecal microbiota of a stool sample. In certain aspects, a preparation of non-selected fecal bacteria comprises all or substantially all of the species of a fecal microbiota of a stool sample. In certain aspects, a preparation of non-selected fecal bacteria comprises all or substantially all of the genera of a fecal microbiota of a stool sample. In certain aspects, a preparation of non-selected fecal bacteria comprises all or substantially all of the phyla of a fecal microbiota of a stool sample. Therefore, such non-selective fecal microbiota can substantially resemble microbial constituents and the bacterial population structure found in such fecal sample.

In an aspect, a preparation of uncultured fecal bacteria comprises at least 2, 5, 10, 20, 30, 40, 50, 100, 200, 300, 400, 500, or 600 bacterial species or strains. In another aspect, a preparation of uncultured fecal bacteria comprises between 2 and 5, 5 and 10, 10 and 20, 20 and 30, 30 and 40, 40 and 50, 50 and 60, 60 and 100, 100 and 200, 200 and 300, 300 and 400, 400 and 500, or 500 and 600 bacterial species or strains.

In an aspect, a preparation of uncultured fecal bacteria and/or non-selected fecal bacteria does not comprise an antibiotic resistant population of bacteria.

In another aspect, the preparation of a composition comprising uncultured fecal bacteria can involve steps that select for a particular, type, state, or taxonomic category of bacteria (e.g., by deliberate removal of certain strains of bacteria, treatment of the population with a selective agent such as ethanol or chloroform, and/or screening of the bacteria for the ability to produce a metabolite at or above a threshold level).

Herein uncultured fecal bacteria are distinguished from a single, purified strain of bacteria such as a bacterial isolate. As used herein, “bacterial isolate” refers to an isolated or purified group of substantially genetically identical bacterial cells generated by proliferation via binary fission from a single predecessor bacterial cell (e.g., by culturing the bacteria). Typically, a bacterial isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. In one aspect, a bacterial isolate is isolated or cultured from the bacterial strains of a fecal microbiota of a subject. Once isolated, in some aspects, a bacterial isolate can be grown as a pure culture of cells; in other aspects, multiple bacterial isolates can be grown simultaneously in the same vessel as a mixed culture. It will be understood that a “bacterial isolate” is a specific purified (e.g. by culturing) form of a bacterial strain. Herein the term “bacterial strain” is typically used herein to refer to bacterial cells of a particular genotype that are in an unpurified, uncultured state (e.g. as they exist in a fecal microbiota or in a preparation of uncultured fecal bacteria). Thus a bacterial strain can be cultured as a bacterial isolate. The term “substantially genetically identical” refers to the very high (e.g. >99.9%) genetic identity shared by different cells in uncontaminated pure compositions of bacterial isolates, owing to their proliferation from a common predecessor, but accounts for minor genetic dissimilarity between cells due to accumulations of relatively rare mutations. Generally, a bacterial isolate is synonymous with a pure culture of bacterial cells. Typically, herein a bacterial isolate consists of non-pathogenic bacteria. In an aspect, a bacterial isolate can be a probiotic, or an ingredient in a probiotic.

As used herein, the term “bacterial cocktail”, sometimes called a “bacterial consortium” or “synthetic bacterial mixture”, refers to an engineered mixture of bacteria comprising a defined consortium of multiple bacterial isolates. The term “defined consortium of multiple bacterial isolates” means that the bacterial cocktail contains two or more bacterial isolates, and that the identity of each bacterial isolate in the cocktail is known, and thus the cocktail can be consistently produced (e.g. by combining isolated bacterial strains) to have a stable composition and properties across separate batches. Herein “identity” of a bacterial isolate can refer to any characteristic of the isolate that uniquely identifies the isolate as different from one or more other bacterial isolates or bacterial strains. Examples of identifying characteristics of a bacterial isolate include nucleotide sequences such as a 16S rRNA sequence, the sequence of one or more coding or non-coding regions of a nucleic acid, and entire genome sequences, levels of gene expression, physiological or metabolic traits, or anatomical traits such as staining pattern or cell wall characteristics.

As used herein, “bacterial mixture” refers to an engineered composition comprising viable bacterial cells. In some aspects, a bacterial mixture comprises one or more non-pathogenic bacterial isolates. In some aspects, a bacterial mixture comprises a preparation of uncultured fecal bacteria. In some aspects, a bacterial mixture comprises both of one or more non-pathogenic bacterial isolates and a preparation of uncultured fecal bacteria.

As used herein, the term “relative abundance” refers to relative representation of an organism of a particular kind (e.g., a bacterial strain, species, or genus) relative to all organisms of similar nature in a certain community (e.g., a preparation of uncultured fecal bacteria or a bacterial mixture). Relative abundance is calculated by dividing the number of an organism of a particular kind by the total number of all organisms of similar nature in a certain community. In an aspect, relative abundance is measured by qPCR comparing PCR products generated with 16S primers targeting specific bacterial strains of interest against PCR products generated with universal primers targeting all 16S sequences. See e.g., Chu, N., et al., “Profiling living bacteria informs preparation of fecal microbiota transplantations.” PLoS One 12(1): 1-16 (2017). In another aspect, the relative abundance is measured based on the number of sequence reads detected via high-throughput sequencing. Unless specified otherwise, a bacterial relative abundance mentioned herein is measured via high-throughput sequencing. In a further aspect, propidium monoazide (PMA) is used to differentiate between viable and dead fecal microbes as shown in Chu et al., PLoS One 12(1): 1-16 (2017).

As used herein, “bacteria,” “bacterium,” and “archaea” refer to single-celled prokaryotes that lack membrane bound nuclei and lack organelles.

As used herein, “colony forming units” (cfu) refers to an estimate of the number of viable microorganism cells in a given sample.

As used herein, “viable” means possessing the ability to multiply.

As used herein, “fecal bacteria” refers to bacteria that can be found in fecal matter.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

Disclosed herein are methods of treating a subject having a condition, disorder or disease related to or caused by a dysbiosis in an intestinal microbiota. Herein “dysbiosis” refers to an imbalance or perturbation in the structure of an intestinal microbial community relative to a healthy intestinal gut flora. In certain aspects such imbalance or perturbation can predispose the subject to development of a particular condition, disorder, or disease, for example an infection, autoimmune disease, allergy or neurological disease.

In an aspect, disclosed herein is a method to treat a patient having a dysbiosis of an intestinal microbiota by delivering to an intestine of the patient one or more bacterial strains. In an aspect, the one or more bacterial strains are present in a bacterial mixture comprising a preparation of uncultured fecal bacteria prepared from a stool of a healthy donor. In an aspect, at least one of the one or more bacterial strains in the bacterial mixture administered to the subject engraft in the intestine of the subject. Herein “engraft” or “engraftment” refers to the stable presence (often accompanied by growth) of a bacterial strain that has been introduced into the intestine of a subject (e.g. by administering to the subject a pharmaceutical composition described herein comprising a preparation of uncultured fecal bacteria comprising the bacterial strain). For example, a bacterial strain can engraft in the intestine of a subject administered the bacterial strain in the form of an FMT (e.g. orally or by enema). In an aspect, engraftment of a bacterial strain introduced into the intestine of a patient can be detected, measured or determined longitudinally, or over time, by identifying a difference in the abundance of the bacterial strain in a fecal sample of the subject before and after administration of the bacterial strain to the subject. In an aspect, the bacterial strain introduced into the subject may be absent from the intestine of the subject prior to the administration. In another aspect, the bacterial strain introduced into the subject may be present in the intestine of the subject prior to the administration, but undetectable or showing an increased abundance following the administration of the bacterial strain. In an aspect, the engraftment of a bacterial strain is detected, measured or determined using values from a plurality of engraftment metrics capable of distinguishing the bacterial strain from other bacterial strains (e.g. of the same species) in a fecal microbiota of the subject or in the preparation of uncultured fecal bacteria administered to the subject in a pharmaceutical composition. In an aspect, at least one of the plurality of metrics is based on one or more parameters of a metagenomics-based DNA sequence of a fecal microbiota of the subject or a preparation of uncultured fecal bacteria administered to the subject in a pharmaceutical composition, as described herein. In certain aspects, engraftment is determined by identifying an increase in abundance of a bacterial strain administered to an intestine of the subject after at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14, days, at least 1 week, at least 2 weeks, at least 3 weeks, at least 4 weeks, at least 5 weeks, at least 6 weeks, at least 7 weeks, at least 8 weeks, at least 1 month, at least 2 months, at least 3 months, at least 4 months, at least 5 months, at least 6 months, or greater than 6 months following administration of the bacterial strain to the subject.

In an aspect, disclosed herein is a method of treating a dysbiosis of an intestinal microbiota in a subject by administering to the subject a pharmaceutical composition (e.g. comprising a preparation of uncultured fecal bacteria prepared from a stool of a healthy human donor); identifying an engraftment status in the intestine of the subject of one or more bacterial strains following said administering; and administering a second dose of the pharmaceutical composition to the subject based on the engraftment status. Herein “engraftment status” refers to the presence or absence of engraftment of a bacterial strain in the intestine of a subject administered the bacterial strain (e.g. in the form of a bacterial mixture comprising a preparation of uncultured fecal bacteria). In an aspect, the engraftment status of a bacterial strain can be negative or positive. A “negative engraftment status” refers to the absence of engraftment of the bacterial strain in the intestine of the subject following its administration. For example, one or more DNA sequence-based metrics can be used (e.g. by incorporating the one or more DNA sequence-based metrics into a machine learning model or algorithm) to determine that the bacterial strain did not engraft after being administered. In an aspect, a second dose of a pharmaceutical composition comprising the bacterial strain can be administered to the subject when the engraftment status of the strain is negative following administration of a first dose of the composition; that is, when a particular bacterial strain does not engraft in the intestine of the subject following the first dose. A “positive engraftment status” refers to the presence of engraftment of a bacterial strain in the intestine of a subject following administration of the strain.

In an aspect, assessing the engraftment status comprises screening a subject for a disrupted or perturbated fecal microbiota by determining or obtaining a value for a microbiome disruption index (MDI) corresponding to the subject's fecal microbiota prior to and/or after administering to the subject a pharmaceutical composition comprising a fecal microbiota or a preparation of uncultured fecal bacteria as described in U.S. patent application Ser. No. 16/667,301 (hereby incorporated by reference herein in its entirety).

Disclosed herein is a method of treating a subject having a dysbiosis of an intestinal microbiota, comprising administering a pharmaceutical composition comprising a preparation of uncultured fecal bacteria to the subject, wherein the fecal bacteria are screened for pathogens; and determining an engraftment status of a bacterial strain in the intestine of the subject following administration of the preparation of uncultured fecal bacteria.

In certain aspects, determining an engraftment status of a bacterial strain comprises metagenomic DNA sequencing of a fecal microbiota of the subject following administration to the subject of the bacterial strain, and comparing values of one or more parameters derived from the DNA sequence to corresponding values of DNA sequence parameters derived from (i) the uncultured fecal bacteria administered to the subject, or (ii) a fecal microbiota of the subject prior to administration of the bacterial strain. For example, such values of DNA sequence parameters (or attributes) can be derived by comparing the DNA sequence of the fecal microbiota or uncultured fecal bacteria to DNA sequences in a database containing reference genomes. In an aspect, a determination that one or more DNA sequence parameter values from uncultured fecal bacteria administered to the subject are different from the fecal microbiota of the subject post-FMT can be indicative of a negative engraftment status of a bacterial strain. In another aspect, a determination that one or more DNA sequence parameter values of uncultured fecal bacteria administered to the subject are the same in the fecal microbiota of the subject post-FMT can be indicative of a positive engraftment status of a bacterial strain. In another aspect, a determination that one or more values of a DNA sequence parameter of a fecal microbiota of a subject post-FMT are different in a fecal microbiota of the subject pre-FMT can be indicative of a positive engraftment status of a bacterial strain. In an aspect, the metagenomic DNA sequencing of the fecal microbiota and/or uncultured fecal bacteria is by whole genome sequencing. In an aspect, the DNA sequencing is by shotgun sequencing.

In certain aspects, determining an engraftment status of a bacterial strain or species comprises comparing values of one or more DNA sequence parameters of uncultured fecal bacteria administered to a subject to corresponding values from a DNA sequence of a fecal microbiota of stool of the subject collected prior to administration of the uncultured fecal bacteria. In certain aspects, determining an engraftment status of a bacterial strain comprises comparing values of one or more DNA sequence parameters of uncultured fecal bacteria administered to a subject to corresponding values from a DNA sequence of a fecal microbiota from stool collected from a subject following administration of the uncultured fecal bacteria. In certain aspects, determining an engraftment status of a bacterial strain comprises comparing values of one or more DNA sequence parameters of a fecal microbiota from stool collected from a subject prior to administration of a preparation of uncultured fecal bacteria to corresponding values of a DNA sequence from a fecal microbiota collected from the subject following administration of the preparation of uncultured fecal bacteria.

In certain aspects, determining a positive engraftment status of a bacterial strain comprises determining that a value of a DNA sequence parameter of a fecal microbiota from a stool of a subject administered a preparation of uncultured fecal bacteria matches the value of the parameter from a DNA sequence of the uncultured fecal bacteria administered to the subject, and optionally determining that the DNA sequence parameter value is different in a DNA sequence obtained from a fecal microbiota of a stool of the subject collected prior to administration of the preparation of uncultured fecal bacteria.

In certain aspects, determining an engraftment status of a bacterial strain comprises determining values of one or more parameters of a metagenomic DNA sequence obtained from, for example, uncultured fecal bacteria administered to a subject, a fecal microbiota prepared from stool of the subject collected prior to administration of a pharmaceutical composition comprising uncultured fecal bacteria, or a fecal microbiota prepared from stool of the subject collected after administration of a pharmaceutical composition comprising uncultured fecal bacteria. In certain aspects, determining or identifying a value of a parameter of a metagenomic DNA sequence comprises comparing the DNA sequence obtained from a fecal microbiota of the subject or from uncultured fecal bacteria administered to the subject to one more DNA sequences in a database of reference genomes. For example, shotgun sequencing of DNA from a fecal microbiota of a subject or from uncultured fecal bacteria administered to a subject, followed by comparison of the DNA sequence to a database of reference genomes (e.g. database of clustered reference genomes), can generate values for the DNA sequence parameters. In certain aspects, the database of reference genomes is based on PATRIC, and clustered using MIDAS (Nayfach et al. (2016), “An integrated metagenomics pipeline for strain profiling reveals novel patterns of bacterial transmission and biogeography”, Genome Research 26: 1612-1625, the entirety of which is incorporated by reference herein).

Herein a “parameter” of a metagenomic DNA sequence refers to any attribute of the DNA sequence that can be used to provide strain-level resolution in inferring engraftment of a bacterial strain in an intestine of a subject following administration of a bacterial mixture to the subject. Non-limiting examples of parameters whose values can be determined by comparing a DNA sequence obtained from a fecal microbiota or uncultured fecal bacteria to DNA sequences in a database of reference genomes include: core gene SNPs, gene content, core gene SNP diversity (heterozygosity), and species abundance. Herein a “core gene” refers to a gene that is generally present across all strain genomes of a particular bacterial species represented in a database of reference genomes. In general, a bacterial species has a ‘core’ genome (consisting of those DNA sequences and genes present in all strains of the same species) and a ‘flexible’ genome (consisting of those DNA sequences and genes that vary across different strains of the same species). In an aspect, a core gene is a single-copy gene. In one aspect, a core gene can be an AMPHORA core gene, for example as described in Wu and Eisen (2008), “A simple, fast, and accurate method of phylogenomic interference” Genome Biology, 9(10): R151, the entirety of which is incorporated by reference herein. Herein “gene content” of a bacterial strain refers to the presence or absence of genes of that strain in the flexible genome. Herein “species abundance” represents the average coverage across all core genes of the species, e.g. the AMPHORA core genes for a species.

In an aspect, disclosed herein is a method of detecting an engraftment of a bacterial strain, the method comprising comparing a value of a parameter of a metagenomic DNA sequence (e.g. determined by comparing the DNA sequence to DNA sequences in a database of reference genomes) obtained from a fecal microbiota (e.g. of a subject pre- post-FMT) or preparation of uncultured fecal bacteria (e.g. prepared from stool of a donor) to a value of a second parameter of a second DNA sequence (e.g. determined by comparing the second DNA sequence to DNA sequences in the database of reference genomes) obtained from a different fecal microbiota or the preparation of uncultured fecal bacteria. Such comparisons can give rise to values of engraftment metrics that can be used to distinguish (e.g. via incorporation of the engraftment metric values into a machine learning model) between strains of bacterial species to determine an engraftment status of a bacterial strain. Herein a “comparison-based engraftment metric” refers to an engraftment metric that is derived by comparing a value of a metagenomic DNA sequence parameter between two DNA sequences, where the two DNA sequences are different and selected from the group consisting of a DNA sequence of a fecal microbiota of a stool of a subject collected pre-FMT, a DNA sequence of a fecal microbiota of a stool of a subject collected post-FMT, and a DNA sequence of the uncultured fecal bacteria administered to the subject in the FMT. A comparison-based engraftment metric is informative about the similarity of a DNA sequence parameter between at least two different environmental states (post-FMT, pre-FMT, or the material administered in the FMT). Accordingly, a comparison-based engraftment metric is representative of attributes from at least two different metagenomic DNA sequences. Typically the comparison is between (i) a DNA sequence parameter value of a fecal microbiota collected from a subject post-FMT versus a DNA sequence parameter value of a fecal microbiota collected from the subject pre-FMT, or (ii) a DNA sequence parameter value of a fecal microbiota collected from a subject post-FMT versus a DNA sequence parameter value of uncultured fecal bacteria administered to the subject.

Non-limiting examples of comparison-based engraftment metrics include: (i) core gene SNP similarity between fecal microbiota of the subject collected before and after administration of a preparation of uncultured fecal bacteria; (ii) core gene SNP similarity between a fecal microbiota of the subject collected after administration of a preparation of uncultured fecal bacteria and the uncultured fecal bacteria administered to the subject; (iii) core gene SNP specificity between fecal microbiota of the subject collected before and after administration of a preparation of uncultured fecal bacteria (specificity defined as similarity normalized by similarity to unrelated samples); (iv) core gene SNP specificity between a fecal microbiota of the subject collected after administration of a preparation of uncultured fecal bacteria and the uncultured fecal bacteria administered to the subject; (v) gene content similarity between fecal microbiota of the subject collected before and after administration of a preparation of uncultured fecal bacteria; (vi) gene content similarity between a fecal microbiota of the subject collected after administration of a preparation of uncultured fecal bacteria and the uncultured fecal bacteria administered to the subject; (vii) gene content specificity between fecal microbiota of the subject collected before and after administration of a preparation of uncultured fecal bacteria; and (viii) gene content specificity between a fecal microbiota of the subject collected after administration of a preparation of uncultured fecal bacteria and the uncultured fecal bacteria administered to the subject.

In another aspect, the methods of detecting engraftment disclosed herein use an engraftment metric that is not derived by comparing parameters of DNA sequences between fecal microbiota or uncultured fecal bacteria. Instead, the metagenomic DNA sequence parameter (e.g. the value of which is determined by comparing the DNA sequence to sequences in a reference database) is used directly (e.g. in a machine learning model) as an engraftment metric to distinguish between strains of bacterial species to determine an engraftment status of a bacterial strain. Such non-comparison-based engraftment metrics are representative of or embody an attribute from only one metagenomic DNA sequence (e.g., deciphered via comparison of the DNA sequence to reference genomes in a database). Non-limiting examples of such non-comparison-based engraftment metrics include (i) core gene SNP diversity (heterozygosity) in a fecal microbiota of the subject collected prior to administration of a preparation of uncultured fecal bacteria; (ii) core gene SNP diversity (heterozygosity) of a fecal microbiota of the subject collected after administration of the preparation of uncultured fecal bacteria; (iii) core gene SNP diversity (heterozygosity) in the preparation of uncultured fecal bacteria administered to the subject; (iv) species abundance in a fecal microbiota of the subject collected prior to administration of a preparation of uncultured fecal bacteria; (v) species abundance in a fecal microbiota of the subject collected after administration of the preparation of uncultured fecal bacteria; and (vi) species abundance in the preparation of uncultured fecal bacteria administered to the subject.

In certain aspects, determining an engraftment status of a bacterial strain following administration to a subject of a preparation of uncultured fecal bacteria comprising the bacterial strain comprises (a) sequencing DNA from (i) a fecal microbiota of the subject collected prior to administration of the preparation of uncultured fecal bacteria; (ii) a fecal microbiota of the subject collected after administration of the preparation of uncultured fecal bacteria; and (iii) the uncultured fecal bacteria; (b) comparing the DNA sequences to sequences in a reference database to generate values for a plurality of parameters for each DNA sequence (e.g. core gene SNP, gene content, core gene SNP diversity (heterozygosity), and species abundance); (c) determining values for a plurality of engraftment metrics based on the values of the DNA sequence parameters (e.g. a plurality of engraftment metrics selected from the group consisting of comparison-based-metrics (i) to (viii) and non-comparison-based metrics (i) to (vi)); and (d) using the plurality of engraftment metrics to determine an engraftment status of the bacterial strain (e.g. by applying the plurality of engraftment metrics to a machine learning model as described herein).

For example, where a bacterial strain that is present in a preparation of uncultured fecal bacteria fails to engraft following administration of the preparation to a subject, a particular pattern of single nucleotide polymorphisms (SNPs) in a DNA sequence of a core gene of the bacterial strain (i.e. relative to DNA sequences of the core genes of the strain's species archived in a database of reference genomes) may be present in a DNA sequence obtained from the preparation of uncultured fecal bacteria, but absent from a DNA sequence obtained from a fecal microbiota of a stool collected from the subject after administration of the preparation (i.e. since the bacterial strain failed to engraft). In such a case, certain comparison-based SNP-dependent metrics (e.g. core gene SNP similarity between a fecal microbiota of the subject collected after administration of a preparation of uncultured fecal bacteria and the uncultured fecal bacteria administered to the subject) can be used to identify that the bacterial strain failed to engraft. Due to the potentially close genetic similarity of different bacterial strains in the fecal microbiota of the subject, the use of values from a plurality of engraftment metrics increases the likelihood that the methods described herein will accurately determine the engraftment status of the strain.

It will be understood that each engraftment metric is representative of, or embodies, an attribute or parameter of a DNA sequence from at least one of a fecal microbiota of a pre-FMT subject, a fecal microbiota of a post-FMT subject, and uncultured fecal bacteria administered to the subject in the FMT. In certain aspects, an engraftment metric can be representative of attributes from at least two DNA sequences selected from the group consisting of a fecal microbiota of a pre-FMT subject, a fecal microbiota of a post-FMT subject, and uncultured fecal bacteria administered to the subject in the FMT. In an aspect, a comparison-based engraftment metric (e.g. comparison-based engraftment metrics (i) to (viii)) is representative of attributes from two different DNA sequences selected from the group consisting of a fecal microbiota of a pre-FMT subject, a fecal microbiota of a post-FMT subject, and uncultured fecal bacteria administered to the subject in the FMT. For example, comparison-based engraftment metric (i) (core gene SNP similarity between fecal microbiota of the subject collected before and after administration of a preparation of uncultured fecal bacteria) is representative of an attribute of a DNA sequence from a fecal microbiota of a stool of the subject collected pre-FMT, and an attribute of a DNA sequence from a fecal microbiota of a stool of the subject collected post-FMT. In another example, comparison-based engraftment metric (ii) (core gene SNP similarity between a fecal microbiota of the subject collected after administration of a preparation of uncultured fecal bacteria and the uncultured fecal bacteria administered to the subject) is representative of an attribute of a DNA sequence from a fecal microbiota of a stool of the subject collected post-FMT, and an attribute of a DNA sequence from the uncultured fecal bacteria administered to the subject in the FMT. In another aspect, an engraftment metric used herein can be representative of or embody an attribute from only a single metagenomic DNA sequence.

In an aspect, a single engraftment metric is used to determine an engraftment status of a bacterial strain. In an aspect, two or more engraftment metrics are used to determine an engraftment status of a bacterial strain. In an aspect, three or more, four, or more, five or more, six or more, seven or more, eight or more nine or more, ten or more, eleven or more, twelve or more, thirteen or more, fourteen or more, fifteen or more, sixteen or more, seventeen or more, eighteen or more, nineteen or more, or twenty or more engraftment metrics are used to determine an engraftment status of a bacterial strain. In an aspect, only a comparison-based engraftment metric (representative of attributes from at least two metagenomic DNA sequences) is used in a method described herein to determine an engraftment status of a bacterial strain. In an aspect, only a non-comparison engraftment metric (representative of an attribute from only one metagenomic DNA sequence) is used in a method described herein to determine an engraftment status of a bacterial strain. In an aspect, both a comparison-based engraftment metric and a non-comparison-based engraftment metric is used in a method described herein to determine an engraftment status of a bacterial strain.

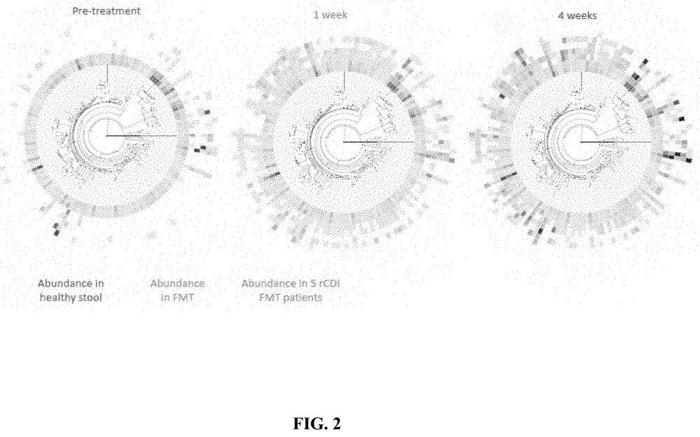

In another aspect, an engraftment metric for use in determining an engraftment status of a bacterial strain can comprise a marker gene abundance. Herein “marker gene abundance” refers to the abundance of genes in a bacterial mixture that are markers for a particular bacterial species. Typically marker gene abundances are used to quantify the species-level composition of a sample, as all strains within a given species contribute to the total abundance of that species' marker genes. For indications such as recurrent C. difficile infection, where patients have severe dysbiosis and are missing many taxa typically found in healthy microbiomes, measurement of marker gene abundance can be effective at identifying new species engrafting from FMT. For example, comparisons across patients receiving the same donor material can reveal species-specific PK, showing that post-FMT patients' gut microbiomes are not simple mixtures of the pre-treatment and donor communities (see FIG. 2). In other aspects, marker gene abundance is not used as an engraftment metric to measure an engraftment status of a bacterial strain.

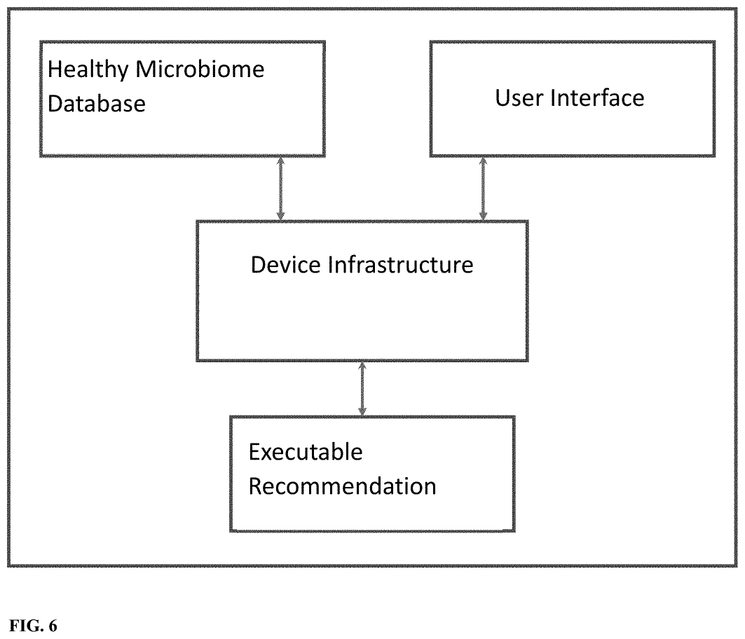

Determining an engraftment status of a bacterial strain following administration of a pharmaceutical composition comprising the bacterial strain can comprise a computer-implemented step (e.g. using the computer illustrated at FIG. 6 herein) that includes processing the values of one or more engraftment metrics with a machine learning model to identify a likelihood of engraftment of the bacterial strain. In an aspect, the model can be a trained machine learning model. For example, the model can be trained with values of engraftment metrics derived from fecal microbiota of control subjects (e.g. from values of DNA sequence parameters of the fecal microbiota, which can be determined by comparing the DNA sequences to a database of reference genomes).

In an aspect, a machine learning training control can be constructed using metagenomic DNA sequencing data from fecal microbiota of stools collected from patients having a dysbiosis of an intestinal microbiota (e.g. related to a disorder) and control subjects not having the dysiosis. In an aspect, the disorder is Inflammatory Bowel Disease (IBD). For example, values of one or more engraftment metrics (i.e. for training the machine learning model) representing a positive control can be derived from (i) a fecal microbiota of a stool from a patient having IBD (representing a subject prior to administration of a pharmaceutical composition comprising a preparation of uncultured fecal bacteria) and (ii) fecal microbiota from two or more stools taken at different time points from a healthy (non-IBD-diagnosed) individual (such that one of the fecal microbiota represents uncultured fecal bacteria administered to the subject and the other fecal microbiota represents a microbiota of a subject administered a preparation of the uncultured fecal bacteria). The values of DNA sequence parameters derived by comparing metagenomic data from such fecal microbiota and uncultured fecal bacteria to sequences in a database of reference genomes can be used to generate values for one or more engraftment metrics as described herein. Such a pattern of engraftment metric values is representative of the successful engraftment of bacterial strains in the intestine of a subject following administration of the strains in a preparation of uncultured fecal bacteria, and can be inputted into a machine learning model to train the model to associate the values with engraftment of the strains, i.e. a positive engraftment status.

Conversely, a negative control for machine learning training can include (i) a fecal microbiota from a stool of a healthy individual (representing the uncultured fecal bacteria administered to a subject); and (ii) fecal microbiota from two or more stools taken at different time points from a patient having IBD (such that one of the fecal microbiota represents a microbiota of a patient prior to administration of the uncultured fecal bacteria and the other fecal microbiota represents a microbiota of the subject following administration of the preparation of uncultured fecal bacteria). As with the positive control, the values of DNA sequence parameters derived by comparing metagenomic data from such fecal microbiota and uncultured fecal bacteria to sequences in a database of reference genomes can be used to generate values for one or more engraftment metrics. Such a pattern of engraftment metric values is representative of the unsuccessful engraftment of bacterial strains in the intestine of a subject following administration of the strains in a preparation of uncultured fecal bacteria, and can be inputted into a machine learning model to train the model to associate the values with a negative engraftment status.

A machine learning model can be trained using metagenomic data from subjects having a gut dysbiosis related to any disorder, disease or condition described herein.

An advantage of a machine-learning approach to identify engraftment status based on values of metagenomic-based engraftment metrics described herein is the ability to use multiple metrics as a means to characterize a bacterial strain in a fecal microbiota. As a result, the method provides a greater likelihood that an engraftment status readout is accurate, and therefore a better diagnosis of the current status of a subject's gut microbiota. Such an approach is particularly advantageous over methods which rely on a single metric (e.g. SNPs) or a few metrics without machine learning, which are incapable of single-strain resolution and thus less likely to accurately determine engraftment.

The present approach allows benchmarking of strain-level donor-patient similarities against (1) patient versus unrelated samples; (2) patient post-FMT versus patient pre-FMT, and (3) the expected variation between longitudinal samples from the same individual. The approach also uses gene content and species abundances in addition to SNP information.

In an aspect, a machine learning model or algorithm is Random Forest, as implemented in the publicly-available Python package sklearn.

In another aspect, an engraftment status of one or more bacterial strains is determined by a machine learning algorithm on a computer or computing device, such as that shown in FIG. 6. In another aspect, assessment of an engraftment status of one or more bacterial strains or species is determined by a computer device for implementing a MDI-based microbiome prediction as provided in U.S. application Ser. No. 16/667,301 (incorporated herein by reference).

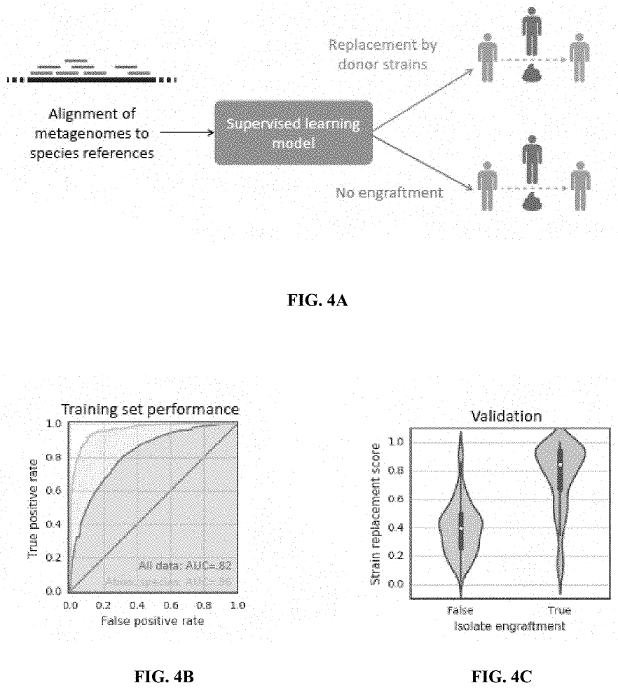

In an aspect, a method of treating a subject with a disorder associated with a dysbiosis of an intestinal microbiota with a composition described herein comprises determining an engraftment status of one or more bacterial strains following administration to the subject of a pharmaceutical composition comprising the one or more bacterial strains (e.g. a bacterial mixture comprising a preparation of uncultured fecal bacteria comprising the bacterial strain and/or a bacterial isolate corresponding to the bacterial strain). In an aspect, a determination of a negative engraftment status for a bacterial strain (see e.g. “false” isolate engraftment in FIG. 4C) in an intestine of the subject following the administration of a pharmaceutical composition comprising the bacterial strain can be used as a basis to re-administer the pharmaceutical composition (e.g. at the same dose or an increased dose) to the subject. In another aspect, a determination of a negative engraftment status for a bacterial strain following administration of a pharmaceutical composition comprising a preparation of uncultured fecal bacteria comprising the bacterial strain can be used as a basis to supplement or ‘spike’ the preparation of uncultured fecal bacteria with the bacterial strain, i.e. to produce or manufacture a bacterial mixture comprising the uncultured fecal bacteria supplemented with the bacterial strain. In an embodiment, the uncultured fecal bacteria can be supplemented with a cultured bacterial isolate corresponding to the bacterial strain. The resulting bacterial mixture, comprising the uncultured fecal bacteria enriched for the bacterial strain, can then be administered in a pharmaceutical composition to the subject. In another aspect, a determination of a negative engraftment status for a bacterial strain following administration of a pharmaceutical composition comprising a preparation of uncultured fecal bacteria comprising the bacterial strain can be used as a basis to re-administer to the subject the preparation of uncultured fecal bacteria, and separately administer the bacterial strain. For example, the preparation of uncultured fecal bacteria can be re-administered to the subject in a first pharmaceutical composition, and the bacterial strain can be administered to the subject in a second composition, e.g. as a bacterial isolate in the form of a probiotic. The composition comprising the bacterial isolate can be administered to the subject, before, after or simultaneously with the pharmaceutical composition comprising the preparation of uncultured fecal bacteria. Alternatively, a determination that the bacterial strain did not engraft can be used as a basis to administer only the bacterial strain (e.g. as a bacterial isolate in the form of a probiotic), without re-administering the preparation of uncultured fecal bacteria.

In an aspect, a method comprises determining that a bacterial strain did not engraft in the intestine of a subject (i.e. determining a negative engraftment status) when a likelihood of engraftment of the bacterial strain (e.g. generated via a trained machine learning model) is less than 50%, less than 40%, less than 30%, less than 20%, less than 10%, less than 5%, less than 4%, less than 3%, less than 2%, less than 1%, or 0%.

In an aspect, a method comprises determining that a bacterial strain did engraft in the intestine of a subject (i.e. determining a positive engraftment status) when a likelihood of engraftment of the bacterial strain (e.g. generated via a trained machine learning model) is greater than 50%, greater than 60%, greater than 70%, greater than 80%, greater than 85%, greater than 90%, greater than 95%, greater than 96%, greater than 97%, greater than 98%, greater than 99%, or 100%.

In an aspect, a pharmaceutical composition described herein comprises a bacterial strain desirable to engraft in the intestine of a subject administered the composition. In an aspect, the composition comprises a preparation of uncultured fecal bacteria. In an aspect, the composition comprises a bacterial isolate. In an aspect, the composition comprises a bacterial mixture comprising a preparation of uncultured fecal bacteria supplemented with a bacterial isolate. In an aspect, a method comprises administering to a subject a pharmaceutical composition comprising a bacterial mixture, wherein one or more bacterial strains in the bacterial mixture engrafts in an intestine of the subject. In an aspect, the bacterial strain that engrafts in the intestine of the subject is from a taxonomic group selected from the group consisting of Firmicutes, Clostridium, Bacteroides, Anaerostipes, Anaerotruncus, Flavonifractor, Coprococcus, Lactobacillus, Lachnospiraceae, Eubacterium, Roseburia, Faecalibacterium, Akkermansia, Alistipes, Clostridium coccoides, Clostridium scindens, Clostridium symbiosum, Clostridium intestinale, Clostridium bifermentans, Clostridium indolis, Bacteroides caccae, Bacteroides coprophilus, Bacteroides distasonis, Bacteroides fragilis, Bacteroides stercoris, Bacteroides vulgatus, Bacteroides cellulosilyticus, Anaerostipes butyraticus, Anaerostipes caccae, Anaerostipes hadrus, Anaerotruncus colihominis, Flavonifractor plautii, Coprococcus comes, Coprococcus eutactus, Coprococcus catus, Lactobacillus plantarum, Lactobacillus casei, Lactobacillus acidophilus, Lactobacillus reuteri, Lactobacillus fermentum, Eubacterium aggregans, Eubacterium brachy, Eubacterium limosum, Eubacterium rectale, Eubacterium eligens, Eubacterium nodatum, Roseburia hominis, Roseburia intestinalis, Roseburia faecis, Roseburia inulinivorans, Faecalibacterium prausnitzii, Akkermansia mucinophila, Alistipes finegoldii, Alistipes shahii, and Alistipes onderdonkii, and a combination thereof.

An engraftment status of one or more bacterial strains or species disclosed herein can be used in a method of treating, preventing or inhibiting a variety of disorders, diseases, conditions or indications caused by or related to a dysbiosis of an intestinal microbiota. For example, an indication of engraftment of a bacterial strain can be used as a marker for the successful treatment of the disorder. Alternatively, an indication that a bacterial strain did not engraft can be used as a basis for administering a second dose of a composition described herein (or as a basis to administer the bacterial strain) to the subject having the disorder, disease, condition or indication. Non-limiting examples of disorders, diseases, conditions or indications contemplated herein as targets of pharmaceutical compositions comprising live, non-pathogenic fecal bacteria include Acne, AIDS Enteropathy, AIDS-related Gastroenteritis, alopecia totalis, Alzheimer's Disease, amyloidosis, amyotrophic lateral sclerosis, ankylosing spondylitis, anorexia, antibiotic associated colitis, Asperger's syndrome, attention deficit disorder (ADD), attention deficit hyperactivity disorder (ADHD), autism spectrum disorder (ASD), Behcet's Syndrome, chronic Clostridium difficile infection (CDI), chronic constipation, chronic depression, chronic fatigue syndrome (CFS), chronic idiopathic pseudo obstructive syndrome, chronic inflammation demyelinating polyneuropathy, chronic nausea, chronic urticaria, coeliac disease, collagenous colitis, colonic polyps, constipation predominant FBD, Crohn's disease, cryptogenic cirrhosis, cyclic vomiting, dermatitis herpetiformis, diabetes, familial mediterranean fever, fatty liver, functional bowel disease (FBD), gastro-oesophageal reflux, gillian-barre syndrome, glomerulonephritis, haemolytic uraemic syndrome, halitosis, IBS constipation-predominant, IBS diarrhea/constipation alternating, IBS diarrhea-predominant, IBS pain-predominant, idiopathic thrombocytopenic purpura (ITP), idiopathic/simple constipation, indeterminate colitis, inflammatory bowel disease (IBD), irritable bowel syndrome (IBS), juvenile diabetes mellitus, lyme disease, manic depressive illness, metabolic syndrome, microscopic colitis, migraine, mixed cryoglobulinaemia, mucous colitis, multiple sclerosis, myasthenia gravis, NASH (nonalcoholic steatohepatitis), non-rheumatoid arthritis, non-rheumatoid factor positive arthritis, non-ulcer dyspepsia, norwalk viral gastroenteritis, obesity, obsessive compulsive disorder, pain predominant FBD, Parkinson's disease, polyarteritis, polyposis coli, primary biliary cirrhosis, primary Clostridium difficile infection (CDI), primary sclerosing cholangitis (PSC), pseudomembranous colitis, psychotic disorders, reiter's syndrome, relapsing diverticulitis, Rett syndrome, rheumatoid arthritis, rosacea, rotavirus gastroenteritis, sacroiliitis, schizophrenia, scleroderma, Sjogren's Syndome, small bowel bacterial overgrowth, sudden infant death syndrome (SIDS), systemic lupus erythematosus, ulcerative colitis, upper abdominal fbd, vasculitic disorders, viral gastroenteritis, pre-diabetic syndrome, type I diabetes, type II diabetes, depression, schizophrenia, a mood disorder, vancomycin-resistant Enterococci (VRE) infection, methicillin-resistant Staphylococcus aureus (MRSA) infection, an autoimmune disorder, an infection, an allergy or atopy and a neurological disorder.

In an aspect, the present disclosure provides a method for treating a disorder, disease or condition related to or caused by a dysbiosis of an intestinal microbiota in a subject in need thereof, where the method comprises administering to the subject a pharmaceutically active dose of a pharmaceutical composition comprising live non-pathogenic bacteria (e.g. a preparation of uncultured fecal bacteria). In one aspect, the method comprises administering daily to the subject a pharmaceutically active dose of a therapeutic composition comprising live non-pathogenic fecal bacteria. In one aspect, a pharmaceutical composition is administered to a patient in need thereof at least once daily or weekly for at least two consecutive days or weeks. In one aspect, a pharmaceutical composition is administered at least once daily or weekly for at least 3, 4, 5, 6, 7, 8, 9, 10, 11, 12, 13, 14, or 15 consecutive days or weeks. In another aspect, a pharmaceutical composition is administered at least once daily or weekly for at least 1, 2, 3, 4, 5, 6, 7, 8, 9, 10, 11, or 12 consecutive days or weeks. In one aspect, a pharmaceutical composition is administered at least once daily or weekly for at most 4, 5, 6, 7, 8, 9, 10, 11, 12, 13, 14, 15, 16, 17, 18, 19, or 20 consecutive days or weeks. In another aspect, a pharmaceutical composition is administered at least once daily or weekly for at most 1, 2, 3, 4, 5, 6, 7, 8, 9, 10, 11, or 12 consecutive weeks or months. In a further aspect, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In one aspect, a pharmaceutical composition is administered to a subject having a disorder, disease or condition related to or caused by a dysbiosis of an intestinal microbiota at least twice daily or weekly for at least two consecutive days or weeks. In one aspect, a pharmaceutical composition is administered at least twice daily or weekly for at least 3, 4, 5, 6, 7, 8, 9, 10, 11, 12, 13, 14, or 15 consecutive days or weeks. In another aspect, a pharmaceutical composition is administered at least twice daily or weekly for at least 1, 2, 3, 4, 5, 6, 7, 8, 9, 10, 11, or 12 consecutive days or weeks. In one aspect, a pharmaceutical composition is administered at least twice daily or weekly for at most 4, 5, 6, 7, 8, 9, 10, 11, 12, 13, 14, 15, 16, 17, 18, 19, or 20 consecutive days or week. In another aspect, a pharmaceutical composition is administered at least twice daily or weekly for at most 1, 2, 3, 4, 5, 6, 7, 8, 9, 10, 11, or 12 consecutive weeks or months. In a further aspect, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In one aspect, a pharmaceutical composition is administered to a subject having a disorder, disease or condition related to or caused by a dysbiosis of an intestinal microbiota at least three times daily or weekly for at least two consecutive days or weeks. In one aspect, a pharmaceutical composition is administered at least three times daily or weekly for at least 3, 4, 5, 6, 7, 8, 9, 10, 11, 12, 13, 14, or 15 consecutive days or weeks. In another aspect, a pharmaceutical composition is administered at least three times daily or weekly for at least 1, 2, 3, 4, 5, 6, 7, 8, 9, 10, 11, or 12 consecutive days or weeks. In one aspect, a pharmaceutical composition is administered at least three times daily for at most 4, 5, 6, 7, 8, 9, 10, 11, 12, 13, 14, 15, 16, 17, 18, 19, or 20 consecutive days or weeks. In another aspect, a pharmaceutical composition is administered at least three times daily for at most 1, 2, 3, 4, 5, 6, 7, 8, 9, 10, 11, or 12 consecutive weeks or months. In a further aspect, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In an aspect, an engraftment status of a bacterial strain is determined as described herein after at least one of a first, a second, a third, a fourth, a fifth, a sixth, a seventh, an eighth, a ninth, a tenth, an eleventh, a twelfth, a thirteenth, a fourteenth, or a fifteenth administration of the pharmaceutical composition.

In one aspect, the present disclosure provides a method for treating a subject having a disorder, disease or condition related to or caused by a dysbiosis of an intestinal microbiota, where the method comprises administering orally to the subject a pharmaceutically active dose of a therapeutic composition described herein, where the dose is administered at a dosing schedule of at least once or twice daily or weekly for at least three consecutive days or weeks. In another aspect, a dose is administered at least once, twice, or three times daily or weekly for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating a subject having a disorder, disease or condition related to or caused by a dysbiosis of an intestinal microbiota, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In an aspect, a first dosing schedule comprises administration to a subject of a pharmaceutical composition comprising a first bacterial mixture, and a second dosing schedule comprises administration to the subject of a pharmaceutical composition comprising a second bacterial mixture. For example, a first bacterial mixture can comprise a preparation of uncultured fecal bacteria, and a second bacterial mixture can comprise a cultured bacterial strain/isolate (e.g. a bacterial strain/isolate for which a negative engraftment status has been determined using the methods described herein). In another example, the second bacterial mixture comprises a preparation of uncultured fecal bacteria supplemented or enriched with a bacterial strain/isolate (e.g. a bacterial strain/isolate for which a negative engraftment status has been determined using the methods described herein).

The methods described herein advantageously facilitate the identification of one or more bacterial strains that have not engrafted in the intestine of a subject administered a preparation of uncultured fecal bacteria. In an aspect, once the lack of engraftment of a bacterial strain is detected, that bacterial strain can be administered to the subject in an attempt to facilitate its engraftment. In an aspect, the bacterial strain can be administered to the subject at an increased dosage compared to its dosage in a previous administration to facilitate engraftment. For example, a pharmaceutical composition comprising a preparation of uncultured fecal bacteria can be re-administered to the subject in a second dosage that is higher than the first dosage, if the strain is found to not be engrafted after administration of the first dose. In an aspect, the preparation of uncultured fecal bacteria in the second dose is prepared from a stool of the same donor as the preparation of uncultured fecal bacteria in the first dose. In another aspect, the preparation of uncultured fecal bacteria in the second dose is prepared from a stool of a different donor as the preparation of uncultured fecal bacteria in the first dose. In certain aspects, a community of uncultured fecal bacteria can be selected for administration to the subject in a second dose of the pharmaceutical composition based on the relative abundance of the bacterial strain of interest in the community of the uncultured fecal bacteria. For example, preparations of uncultured fecal bacteria derived from different donors can be screened to identify the relative abundance in the preparation of a bacterial strain of interest, and the preparation with the higher relative abundance of the strain can be selected for administration to the subject in a pharmaceutical composition.

In another aspect, a subject showing a negative engraftment status of a bacterial strain is administered a second dose of a pharmaceutical composition comprising a preparation of uncultured fecal bacteria that is supplemented or spiked with the bacterial strain, e.g. cultured as a bacterial isolate. Such supplementation has the benefit of increasing the relative abundance of the bacterial strain in the pharmaceutical composition, while maintaining the contextual ‘background’ of microbes in the composition, which may facilitate engraftment of the bacterial strain in the intestine.

In an aspect, a pharmaceutical composition comprises a bacterial mixture comprising the bacterial strain of interest (e.g. cultured as a bacterial isolate) and a preparation of uncultured fecal bacteria, such that a relative abundance of viable cells of the bacterial isolate in the bacterial mixture is less than a relative abundance of viable cells of the preparation of uncultured fecal bacteria (i.e., where the bacterial mixture comprises only one bacterial isolate, less than 50% of the viable cells of the bacterial mixture are cells of the bacterial isolate). In an aspect, a relative abundance of viable cells of the bacterial isolate in a bacterial mixture comprising the bacterial isolate and a preparation of uncultured fecal bacteria is less than 50%, less than 45%, less than 40%, less than 35%, less than 30%, less than 25%, less than 20%, less than 15%, less than 10%, less than 5%, less than 3%, or less than 1%.

In an aspect, a pharmaceutical composition comprises a bacterial mixture comprising the bacterial strain of interest (e.g. cultured as a bacterial isolate) and a preparation of uncultured fecal bacteria, such that a relative abundance of viable cells of the preparation of uncultured fecal bacteria in the bacterial mixture is less than a relative abundance of viable cells of the bacterial isolate (i.e., where the bacterial mixture comprises only one bacterial isolate, less than 50% of the viable cells of the bacterial mixture are cells of the preparation of uncultured fecal bacteria). In another aspect, a relative abundance of viable cells of the preparation of uncultured fecal bacteria in a bacterial mixture comprising the preparation of uncultured fecal bacteria and a bacteria isolate is less than 50%, less than 45%, less than 40%, less than 35%, less than 30%, less than 25%, less than 20%, less than 15%, less than 10%, less than 5%, less than 3%, or less than 1%.

In an aspect, a pharmaceutical composition comprises a bacterial mixture comprising the bacterial strain of interest (e.g. cultured as a bacterial isolate) and a preparation of uncultured fecal bacteria, such that a relative abundance of viable cells of the preparation of uncultured fecal bacteria is about equal to a relative abundance of viable cells of the bacterial isolate (i.e., where the bacterial mixture comprises only one bacterial isolate, about 50% of the viable cells of the bacterial mixture are cells of the bacterial isolate, and about 50% of the viable cells of the bacterial mixture are cells of the preparation of uncultured fecal bacteria).

In an aspect, a pharmaceutical composition comprises a bacterial mixture comprising the bacterial strain of interest (e.g. cultured as a bacterial isolate) and a preparation of uncultured fecal bacteria, such that a relative abundance of viable cells of the bacterial isolate is greater than the relative abundance of viable cells of any bacterial strain, any bacterial species, any bacterial genus, any bacterial family, any bacterial order, any bacterial class, or any bacterial phylum in the a preparation of uncultured fecal bacteria.

In another aspect, a subject showing a negative engraftment status of a bacterial strain is not administered a second dose of a preparation of uncultured fecal bacteria. Instead, the subject can be administered a bacterial mixture comprising the bacterial strain (e.g. cultured as a bacterial isolate) in the absence of uncultured fecal bacteria. For example, the bacterial strain can be administered to the subject as a prebiotic.

In some cases, a subject showing a negative engraftment status following administration of a bacterial mixture described herein (e.g. comprising a preparation of uncultured fecal bacteria) can be administered a bacterial strain (e.g. as a cultured bacterial isolate) that is not genetically identical to the unengrafted bacterial strain. For example, a bacterial isolate administered to the subject (e.g. on its own or supplemented into a preparation of uncultured fecal bacteria) can be at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, or at least 99.9% genetically identical to the unengrafted bacterial strain. In an aspect, the genetic identify of the bacterial isolate to the unengrafted bacterial isolate can be determined based on the genetic identity of a 16S rRNA sequence between the strain and the isolate. For example, a bacterial isolate can be administered to the subject after determining that a bacterial strain has not engrafted based on the isolate comprising a 16S rRNA sequence that is at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, or at least 99.9% genetically identical to a 16S rRNa sequence the unengrafted bacterial strain. In other aspects, other measures of genetic identity, such as whole genome sequences, can be used as a basis for selecting a bacterial isolate to administer to a subject in place of or to substitute the lack of the engrafted bacterial strain.

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a human patient. In one aspect, a patient is a male patient. In one aspect, a patient is a female patient. In one aspect, a patient is a premature newborn. In an aspect, a patient is a male premature newborn. In another aspect, a patient is a female premature newborn. In one aspect, a patient is a term newborn. In an aspect, a patient is a male term newborn. In another aspect, a patient is a female term newborn. In one aspect, a patient is a neonate. In one aspect, a patient is an infant. In another aspect, a patient is a male infant. In another aspect, a patient is a female infant. In one aspect, a patient is a toddler. In another aspect, a patient is a male toddler. In another aspect, a patient is a female toddler. In one aspect, a patient is a young child. In one aspect, a patient is a child. In another aspect, a patient is a male child. In another aspect, a patient is a female child. In one aspect, a patient is an adolescent. In one aspect, a patient is a pediatric patient. In another aspect, a patient is a male pediatric patient. In another aspect, a patient is a female pediatric patient. In one aspect, a patient is a geriatric patient. In another aspect, a patient is a male geriatric patient. In another aspect, a patient is a female geriatric patient. In one aspect, a patient is an adult male. In another aspect, the patient is an adult female. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a subject being treated is a patient on a limited diet. In another aspect, a subject being treated is a patient on a non-limited diet. In another aspect, a subject being treated is a patient on a diet comprising animal protein. In another aspect, a subject being treated is a patient on a diet comprising spicy foods. In another aspect, a subject being treated is a patient on a diet comprising high fat food.

In one aspect, a method comprises administering a pharmaceutical composition orally, by enema, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice cream, or a yogurt. In another aspect, a pharmaceutical composition administered herein is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid culture. In another aspect, a pharmaceutical composition is homogenized, lyophilized, pulverized and powdered. It may then be infused, dissolved such as in saline, as an enema. Alternatively the powder may be encapsulated as enteric-coated and/or acid-resistant delayed release capsules for oral administration. In an aspect, the powder may be double encapsulated with acid-resistant/delayed release capsules for oral administration. These capsules may take the form of enteric-coated and/or acid-resistant delayed release microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a pharmaceutical composition administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a bacterial mixture incorporated into a pharmaceutical composition is a lyophilized formulation further comprising a reducing agent. In certain aspects, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfate, sodium metabisulfite, potassium metabisulfite, Glutathione, Methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a bacterial mixture incorporated into a pharmaceutical composition comprises a cryoprotectant. As used herein, a “cryoprotectant” refers to a substance that is added to a formulation in order to protect an active ingredient during freezing. In an aspect, a cryoprotectant comprises, consists essentially of, or consists of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof. In an aspect of the present disclosure, a cryoprotectant can be selected from the group comprising 5% Sucrose; 10% Sucrose; 10% Skim milk; 10% trehalose with 2.5% sucrose; 5% trehalose with 2.5% sucrose; 5% mannitol; 5% mannitol with 0.1% Polysorbate 80; 10% mannitol; 10% mannitol with 0.1% Polysorbate 80; 5% trehalose; 5% trehalose with 0.1% Polysorbate 80; 10% trehalose; and 10% trehaolse with 0.1% Polysorbate 80.

In another aspect, a bacterial mixture incorporated into a pharmaceutical composition comprises a lyoprotectant. As used herein, a “lyoprotectant” refers to a substance that is added to a formulation in order to protect an active ingredient during the drying stage of a lyophilization (also known as freeze-drying) process. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consists essentially of, or consists of, one or more substances mentioned in this paragraph and the paragraph above.

In one aspect, a cryoprotectant or a lyoprotectant comprise an intracellular agent, e.g., DMSO, Glycerol, or PEG, which penetrates inside the cell preventing the formation of ice crystals that could result in membrane rupture. In another aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In one aspect, a pharmaceutical composition administered herein further comprises an acid suppressant, an antacid, an H2 antagonist, a proton pump inhibitor or a combination thereof. In one aspect, a pharmaceutical composition administered herein substantially free of non-living matter. In another aspect, a pharmaceutical composition administered herein substantially free of acellular material selected from the group consisting of residual fiber, DNA, viral coat material, and non-viable material. In another aspect, a pharmaceutical composition administered does not comprise an acid suppressant, an antacid, an H2 antagonist, a proton pump inhibitor or a combination thereof. In yet another aspect, a pharmaceutical composition administered does not comprise an acid suppressant. In another aspect, a pharmaceutical composition administered does not comprise an antacid. In another aspect, a pharmaceutical composition administered does not comprise an H2 antagonist. In another aspect, a pharmaceutical composition administered does not comprise a proton pump inhibitor. In another aspect, a pharmaceutical composition administered does not comprise metoclopramide.

In one aspect, a pharmaceutical composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides. In another aspect, a subject is not pretreated with a prebiotic nutrient prior to treatment with a pharmaceutical composition. In another aspect, the pharmaceutical composition is not supplemented with a prebiotic nutrient.

In one aspect, a method further comprises pretreating a subject with an antibiotic composition prior to administering a bacterial mixture. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a pharmaceutical composition. In another aspect, the pharmaceutical composition is not supplemented with an antibiotic composition. In a further aspect, a method further comprises pretreating a subject with an anti-inflammatory drug prior to administration of a pharmaceutical composition. In yet another aspect, a subject is not pretreated with an anti-inflammatory drug prior to administering a pharmaceutical composition. In another aspect, a pharmaceutical)composition is not supplemented with an anti-inflammatory.

In an aspect of the present disclosure, a method further comprises administering a pharmaceutical composition to a subject in need thereof, without co-administering steroids. In another aspect, the subject has not been previously treated with steroids to treat a dysbiosis. In yet another aspect, the subject is not administered a steroid at least 1, 2, 3, 4, 5, 6, 10, 15, 20, 25, 30, 35, 40, 45, or 50 weeks prior to the administering of a pharmaceutical composition. In a further aspect, the subject is not administered a steroid at least 1, 2, 3, 4, 5, 6, 7, 7, 9, or 10 years prior to the administering of a pharmaceutical composition. In yet another aspect, the subject is not treated with steroids for at least 1, 2, 3, or 4 weeks prior to or after the administering of a pharmaceutical composition (e.g. comprising uncultured fecal bacteria). In another aspect, the subject is not co-treated with drugs to treat conditions of dysbiosis (e.g., Crohn's disease, ulcerative colitis, irritable bowel disease, etc.). in yet another aspect, a subject is not co-treated with thiopurines or 5-aminoscalicylate (5-ASA). In a further aspect, a subject is not co-treated with a corticosteroid, 5-ASA products, immunomodulators, anti-TNFα agents, or other medication prescribed to treat Crohn's disease, ulcerative colitis, irritable bowel syndrome, and irritable bowel disease. in another aspect, a subject is not co-treated with a drug used to treat gastrointestinal disorders.

In an aspect of the present disclosure, a method further comprises administering a pharmaceutical composition to a subject in need thereof, without co-administering nonsteroidal anti-inflammatory drugs. In another aspect, the subject has not been previously treated with nonsteroidal anti-inflammatory drugs to prevent ulcerative colitis flare-ups. In yet another aspect, the subject is not administered a nonsteroidal anti-inflammatory drug at least 1, 2, 3, 4, 5, 6, 10, 15, 20, 25, 30, 35, 40, 45, or 50 weeks prior to the administering of a pharmaceutical composition. In a further aspect, the subject is not administered a nonsteroidal anti-inflammatory drug at least 1, 2, 3, 4, 5, 6, 7, 7, 9, or 10 years prior to the administering of a pharmaceutical composition. In yet another aspect, the subject is not treated with nonsteroidal anti-inflammatory drug for at least 1, 2, 3, or 4 weeks prior to or after the administering of a pharmaceutical composition. In another aspect, the subject is not treated with mesalamine for at least 1, 2, 3, or 4 weeks prior to or after the administering of a pharmaceutical composition comprising a bacterial mixture disclosed herein.

The compositions and methods of the present invention may further comprise one or more prebiotics. A prebiotic is a substrate that is selectively used by a host microorganism to produce a health benefit in a subject. Without wishing to be bound by theory, prebiotics are added to nutritionally supplement bacteria in the microbiome and/or in a microbial composition, e.g., to stimulate the growth or activity of one or more strains of beneficial bacteria. Additionally, the prebiotics may be added to prevent “shock” to bacterial strains subsequent to their isolation or purification, freezing, freeze-drying, spray-drying, reconstitution in solution and the like.

Examples of prebiotics include amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In aspects, a prebiotic can be added (e.g., in dry or liquid forms) to a microbial composition of the present invention.

Alternately, or additionally, a prebiotic can be included (e.g., in dry or liquid forms) in a distinct pharmaceutical composition which lacks a microbial composition of the present invention.

A prebiotic may be provided to a subject before, contemporaneously with, and/or after a pharmaceutical composition comprising a microbial composition of the present invention is administered, either in a pharmaceutical composition comprising the microbial composition or in a pharmaceutical composition lacking a microbial composition.

A prebiotic may be provided in a single dose or in multiple doses. When provided as a single composition, the single composition may comprise a single prebiotic or a mixture of prebiotics. When provided in multiple compositions, each composition may comprise a single prebiotic or a mixture of prebiotics.

As examples, when multiple doses are provided, a first composition comprising a prebiotic may include one specific prebiotic, e.g., inulin, and a second composition may include a second specific prebiotic, e.g., pectin. Alternately, a first composition may include a mixture of prebiotics, e.g., inulin and pectin and a second composition may include different mixture of prebiotics, e.g., inulin and a FOS. A first composition may include a mixture of prebiotics and a second composition may include one specific prebiotic.

The amount of prebiotic provided to a subject/patient and/or included in a composition depends on the specific prebiotic, the specific bacterial strain of beneficial bacteria, and/or the disease state of the subject.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cells or spores. In another aspect, a pharmaceutically active or therapeutic effective dose comprises at most about 10⁵, 10⁶, 10⁷, 10⁸, 10⁹, 10¹⁰, 10¹¹, 10¹², 10¹³, 10¹⁴, or 10¹⁵ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter. In an aspect, a pharmaceutically active or therapeutic effective dose comprises between 10¹⁰ and 10¹² cells. In another aspect, a pharmaceutically active or therapeutic effective dose comprises between 10¹⁰ and 10¹² cells per capsule. In another aspect, a pharmaceutically active or therapeutic effective dose comprises between 10¹¹ and 10¹² cells per capsule. In a further aspect, a pharmaceutically active or therapeutic effective dose comprises between 10⁹ and 10¹² cells per capsule.

In one aspect, a pharmaceutical composition administered herein comprises fecal bacteria. In one aspect, a pharmaceutical composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a pharmaceutical composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, a pharmaceutical composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a pharmaceutical composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, or a combination thereof.

In one aspect, a pharmaceutical composition administered herein comprises a fecal microbiota and/or uncultured fecal bacteria. In another aspect, a preparation of uncultured fecal bacteria is prepared using a treatment selected from the group consisting of ethanol treatment, detergent treatment, heat treatment, irradiation, and sonication. In another aspect, a preparation of uncultured fecal bacteria is prepared using no treatment selected from the group consisting of ethanol treatment, detergent treatment, heat treatment, irradiation, and sonication. In one aspect, manufacture of a preparation of uncultured fecal bacteria involves a separation step selected from the group consisting of density gradients, filtration (e.g., sieves, nylon mesh), and chromatography. In another aspect, manufacture of a preparation of uncultured fecal bacteria used herein involves no separation step selected from the group consisting of density gradients, filtration (e.g., sieves, nylon mesh), and chromatography. In another aspect, \a preparation of uncultured fecal bacteria comprises a donor's entire fecal microbiota. In another aspect, a pharmaceutical composition administered herein comprises a preparation of uncultured fecal bacteria is substantially free of eukaryotic cells from the donor of the cells.

In another aspect, a pharmaceutical composition administered herein comprises a preparation of uncultured fecal bacteria further supplemented, spiked, or enhanced with a fecal microorganism. In one aspect, a preparation of uncultured fecal bacteria is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a pharmaceutical composition administered herein comprises a preparation of uncultured fecal bacteria further supplemented, spiked, or enhanced with a species of Veillonellaceae, Firmicutes, Gammaproteobacteria, Bacteroidetes, or a combination thereof. In another aspect, a pharmaceutical composition administered herein comprises a preparation of uncultured fecal bacteria further supplemented with fecal bacterial spores. In one aspect, fecal bacterial spores are Clostridium spores, Bacillus spores, or both.

In an aspect, a pharmaceutical composition comprises a preparation of uncultured fecal bacteria from a subject selected from the group consisting of a human, a bovine, a dairy calf, a ruminant, an ovine, a caprine, or a cervine. In another aspect, a pharmaceutical composition can be administered to a subject selected from the group consisting of a human, a bovine, a dairy calf, a ruminant, an ovine, a caprine, or a cervine. In an aspect, a pharmaceutical composition is substantially or nearly odourless.

In an aspect, a pharmaceutical composition provided or administered herein comprises a a preparation of uncultured fecal bacteria comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises a preparation of uncultured fecal bacteria comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora.

In a further aspect, a pharmaceutical composition comprises fecal bacteria from at least 1, 2, 3, 4, 5, 6, 7, 8, 9, or 10 different families. In another aspect, a pharmaceutical composition comprises fecal bacteria from at least 11, 12, 13, 14, 15, 16, 17, 18, 19, or 20 different families. In yet another aspect, a pharmaceutical composition comprises fecal bacteria from at least 21, 22, 23, 24, 25, 26, 27, 28, 29, or 30 different families. In a further aspect, a pharmaceutical composition comprises fecal bacteria from at least 31, 32, 33, 34, 35, 36, 37, 38, 39, or 40 different families. In another aspect, a pharmaceutical composition comprises fecal bacteria from at least 41, 42, 43, 44, 45, 46, 47, 48, 49, or 50 different families. In another aspect, a pharmaceutical composition comprises fecal bacteria from between 1 and 10, between 10 and 20, between 20 and 30, between 30 and 40, between 40 and 50 different families. In an aspect, a pharmaceutical composition provided or administered herein comprises a preparation of uncultured fecal bacteria comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a pharmaceutical composition provided or administered herein comprises a preparation of uncultured fecal bacteria comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a pharmaceutical composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In an aspect, “biological material” refers to the living material, e.g., the microbes, eukaryotic cells, and viruses, which are present in the colon of a normal healthy human. In an aspect, a pharmaceutical composition provided or administered herein comprises an extract of human feces where the composition is substantially odorless. In an aspect, a pharmaceutical composition provided or administered herein comprises fecal material or a fecal floral preparation in a lyophilized, crude, semi-purified or purified formulation.

In an aspect, a a preparation of uncultured fecal bacteria in a pharmaceutical composition comprises highly refined or purified fecal flora, e.g., substantially free of non-floral fecal material. In an aspect, a preparation of uncultured fecal bacteria can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a preparation of uncultured fecal bacteria in a pharmaceutical composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a preparation of uncultured fecal bacteria in a pharmaceutical composition comprises a donor's substantially entire or non-selected fecal microbiota, reconstituted fecal material, or synthetic fecal material. In another aspect, the fecal microbiota in a pharmaceutical composition comprises no antibiotic resistant population. In another aspect, a pharmaceutical composition comprises a preparation of uncultured fecal bacteria and is largely free of extraneous matter (e.g., non-living matter including acellular matter such as residual fiber, DNA, RNA, viral coat material, non-viable material; and living matter such as eukaryotic cells from the fecal matter's donor).

In an aspect, a preparation of uncultured fecal bacteria in a pharmaceutical composition used herein is derived from disease-screened fresh homologous feces or equivalent freeze-dried and reconstituted feces. In an aspect, a fresh homologous feces does not include an antibiotic resistant population. In another aspect, a preparation of uncultured fecal bacteria in a pharmaceutical composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a pharmaceutical composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, an exemplary pharmaceutical composition comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a pharmaceutical composition used herein comprises a reconstituted fecal flora consisting essentially of a combination of a purified fecal microbiota and a non-cellular fecal filtrate. In another aspect, a pharmaceutical composition used here comprises a purified fecal microbiota supplemented with one or more non-cellular non-particulate fecal components. In one aspect, a pharmaceutical composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based pharmaceutical composition is used concurrently with a fecal non-cellular filtrate-based pharmaceutical composition. In another aspect, a patient is treated with a first fecal non-cellular filtrate-based pharmaceutical composition before being given a second fecal bacteria-based pharmaceutical composition, or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, giving the patient a fecal bacteria-based pharmaceutical composition to re-establish a functional intestinal microbiome.

In an aspect, a pharmaceutical composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In an aspect, a pharmaceutical composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of an intestinal dysbiosis of a subject. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a pharmaceutical composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a pharmaceutical composition may contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a pharmaceutical composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A pharmaceutical composition used herein can be flavored.

In an aspect, a pharmaceutical composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a pharmaceutical composition can be a drench. In one aspect, a drench is prepared by choosing a saline-suspended form of a pharmaceutical composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a pharmaceutical composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a pharmaceutical composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a pharmaceutical composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a pharmaceutical composition comprises two or more genera selected from the group consisting of Collinsella, Coprococcus, Dorea, Eubacterium, and Ruminococcus. In another aspect, a pharmaceutical composition comprises two or more genera selected from the group consisting of Coprococcus, Dorea, Eubacterium, and Ruminococcus. In a further aspect, a pharmaceutical composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a pharmaceutical composition comprises at least about 10⁵, 10⁶, 10⁷, 10⁸, 10⁹, 10¹⁰, 10¹¹, 10¹², or 10¹³ cfu or total cell count. In another aspect, a pharmaceutical composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a pharmaceutical composition comprises at least about 10⁵, 10⁶, 10⁷, 10⁸, 10⁹, 10¹⁰, 10¹¹, 10¹², or 10¹³ cells or total cell count. In another aspect, a pharmaceutical composition comprises at most about 10⁵, 10⁶, 10⁷, 10⁸, 10⁹, 10¹⁰, 10¹¹, 10¹², 10¹³ or 10¹⁴ cells or total cell count.

In one aspect, a pharmaceutical composition is formulated as an oral capsule, microcapsule, tablet, or pill. In another aspect, a capsule, microcapsule, tablet, or pill is adapted for enteric delivery. In a further aspect, a capsule, microcapsule, tablet, or pill is an enteric capsule, microcapsule, tablet, or pill. In another aspect, a capsule, microcapsule, tablet, or pill comprises an enteric coating, is acid resistant, or both.

In one aspect, an exemplary pharmaceutical composition comprises starting material from a donor. In another aspect, an exemplary pharmaceutical composition comprises material from one or more healthy donors. In yet another aspect, an exemplary pharmaceutical composition comprises starting material from a defined donor pool. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1 year old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a carefully screened donor undergoes a complete medical history and physical exam. Donors are excluded if they have a risk of infectious agents. Additional exclusion criteria comprises the following:

1. Known viral infection with Hepatitis B, C or HIV
2. Known exposure to HIV or viral hepatitis at any time
3. High risk behaviors including sex for drugs or money, men who have sex with men, more than one sexual partner in the preceding 12 months, any past use of intravenous drugs or intranasal cocaine, history of incarceration.
4. Tattoo or body piercing within 12 months.
5. Travel to areas of the world where risk of traveler's diarrhea is higher than the US.
6. Current communicable disease, e.g., upper respiratory viral infection.
7. History of irritable bowel syndrome. Specific symptoms may include frequent abdominal cramps, excessive gas, bloating, abdominal distension, fecal urgency, diarrhea, constipation.
8. History of inflammatory bowel disease such as Crohn's disease, ulcerative colitits, microscopic colitis.
9. Chronic diarrhea.
10. Chronic constipation or use of laxatives.
11. History of gastrointestinal malignancy or known colon polyposis.
12. History of any abdominal surgery, e.g., gastric bypass, intestinal resection, appendectomy, cholecystectomy, etc.
13. Use of Probiotics or any other over the counter aids used by the potential donor for purpose of regulating digestion. Yogurt and kefir products are allowed if taken merely as food rather than nutritional supplements.
14. Antibiotics for any indication within the preceding 6 months.
15. Any prescribed immunosuppressive or anti-neoplastic medications.
16. Metabolic Syndrome, established or emerging. Criteria used for definition here are stricter than any established criteria. These include history of increased blood pressure, history of diabetes or glucose intolerance.
17. Known systemic autoimmunity, e.g., connective tissue disease, multiple sclerosis.
18. Known atopic diseases including asthma or eczema.
19. Chronic pain syndromes including fibromyalgia, chronic fatigue syndrome.
20. Ongoing (even if intermittent) use of any prescribed medications, including inhalers or topical creams and ointments.
21. Neurologic, neurodevelopmental, and neurodegenerative disorders including autism, Parkinson's disease.
22. General. Body mass index >26 kg/ m2, central obesity defined by waste:hip ratio >0.85 (male) and >0.80 (female).
23. Blood pressure >135 mmHg systolic and >85 mmHg diastolic.
24. Skin—presence of a rash, tattoos or body piercing placed within a year, or jaundice
25. Enlarged lymph nodes.
26. Wheezing on auscultation.
27. Hepatomegaly or stigmata of liver disease.
28. Swollen or tender joints. Muscle weakness.
29. Abnormal neurologic examination.
30. Positive stool Clostridium difficile toxin B tested by PCR.
31. Positive stool cultures for any of the routine pathogens including Salmonella, Shigella, Yersinia, Campylobacter, E. coli 0157:H7.
32. Abnormal ova and parasites examination.
33. Positive Giardia, Cryptosporidium, or Helicobacter pylori antigens.
34. Positive screening for any viral illnesses, including HIV 1 and 2, Viral Hepatitis A IgM, Hepatitis surface antigen and core Ab.
35. Abnormal RPR (screen for syphilis).
36. Any abnormal liver function tests including alkaline phosphatase, aspartate aminotransaminase, alanine aminotransferase.
37. Raised serum triglycerides >150 mg/Dl
38. HDL cholesterol <40 mg/dL (males) and <50 mg/dL (females)
39. High sensitivity CRP >2.4 mg/L
40. Raised fasting plasma glucose (>100 mg/dL)

In one aspect, a subject in need thereof is administered a pharmaceutical composition comprising fecal microbiota of multiple carefully screened, healthy donors. In an aspect, a subject is administered a pharmaceutical composition over a dosing period wherein a first dose comprises at least one pharmaceutical composition comprises a preparation of uncultured fecal bacteria of a single donor, and a second dose of a pharmaceutical composition comprises a preparation of uncultured fecal bacteria of a single donor different from the donor of the first dose. In another aspect, a first dose comprises a pharmaceutical composition comprising a preparation of uncultured fecal bacteria of a single donor and a second dose comprises a preparation of uncultured fecal bacteria of a donor pool. The first and second dose do not indicate the order of administration to a subject, but rather that a preparation of uncultured fecal bacteria from separate donors may be used in a non-blended form.

In another aspect, the present disclosure provides for methods for treating a subject in need thereof with capsules containing a pharmaceutical composition comprising a preparation of uncultured fecal bacteria from a single donor. In another aspect, a capsule comprises a pharmaceutical composition comprising a preparation of uncultured fecal bacteria from multiple donors. In one aspect a subject is administered two or more pills comprising a preparation of uncultured fecal bacteria from a single but different donor.

In one aspect, the present disclosure provides for methods for treating a subject in need thereof comprising administering a pharmaceutical composition orally or by infusions through a colonoscope, an enema or via a nasojejunal tube. In another aspect, each administration comprises a pharmaceutical composition comprising a preparation of uncultured fecal bacteria of a single donor similar to or different from a prior administration in a treatment period. In another aspect, a treatment period includes administration of a first dost comprising a pharmaceutical composition comprising a preparation of uncultured fecal bacteria of a single donor and administration of a second dose comprising a pharmaceutical composition comprising a preparation of uncultured fecal bacteria of multiple donors.

The present disclosure provides for the following embodiments:

Embodiment 1. A method for treating a disorder by increasing an abundance of a bacterial strain in an intestine of a subject, the method comprising: administering a therapeutic composition comprising a fecal microbiota of a human donor to the subject, wherein the fecal microbiota comprises a bacterial strain; and administering a second dose of the therapeutic composition to the subject based on an engraftment status of the bacterial strain, wherein the engraftment status is determined based on a value of two or more metrics of the bacterial strain.

Embodiment 2. The method of embodiment 1, wherein at least one of the two or more metrics is derived from a DNA sequence of a fecal microbiota of a stool sample.

Embodiment 3. The method of Embodiment 2, wherein the stool sample is derived from one or more of the donor, the subject prior to said administering a therapeutic composition, and the subject after said administering the therapeutic composition.

Embodiment 4. The method of any one of embodiments 1 to 3, wherein the two or more metrics are selected from the group consisting of: core gene SNP similarity between post-FMT and pre-FMT; core gene SNP similarity between post-FMT and the donor; core gene SNP specificity between post-FMT and pre-FMT (specificity defined as similarity normalized by similarity to unrelated samples); core gene SNP specificity between post-FMT and the donor; gene content similarity between post-FMT and pre-FMT; gene content similarity between post-FMT and the donor; gene content specificity between post-FMT and pre-FMT; gene content specificity between post-FMT and the donor; core gene SNP diversity (heterozygosity) in pre-FMT; core gene SNP diversity (heterozygosity) in post-FMT; core gene SNP diversity (heterozygosity) in the donor; species abundance in pre-FMT; species abundance in post-FMT; and species abundance in the donor.

Embodiment 5. The method of any one of embodiments 1 to 4, wherein the engraftment status is determined based on the values of the two or more metrics by a machine learning algorithm.

Embodiment 6. The method of embodiment 5, wherein the engraftment status comprises a likelihood that the bacterial strain did not engraft based on a result of the machine learning algorithm.

Embodiment 7. The method of any one of embodiments 1 to 6, further comprising determining a second engraftment status of the bacterial strain following said administering a second dose of the composition.

Embodiment 8. The method of embodiment 7, wherein the second engraftment status comprises a likelihood that the bacterial strain did engraft based on a result of the machine learning algorithm.

Embodiment 9. The method of any one of embodiments 1 to 8, wherein the bacterial strain is at least one of a member of Clostridium, a member of Lachnospiraceae, or Bacteroides coprophilus.

Embodiment 10. The method of any one of embodiments 1-9, wherein the fecal microbiota is a substantially complete fecal microbiota.

### EXAMPLES

### Example 1: Engraftment in Dysbiotic Patients

Here engraftment of donor species is determined using marker-gene abundances. For indications such as recurrent C. difficile infection, where patients have severe dysbiosis and are missing many taxa typically found in healthy microbiomes, this approach is effective at identifying new species engrafting from FMT. FIG. 2 shows that comparisons across patients receiving the same donor material reveal species-specific pharmacokinetics, showing that post-FMT patients' gut microbiomes are not simple mixtures of the pre-treatment and donor communities.

### Example 2: Microbiome Changes in Inflammatory Bowel Disease (IBD)

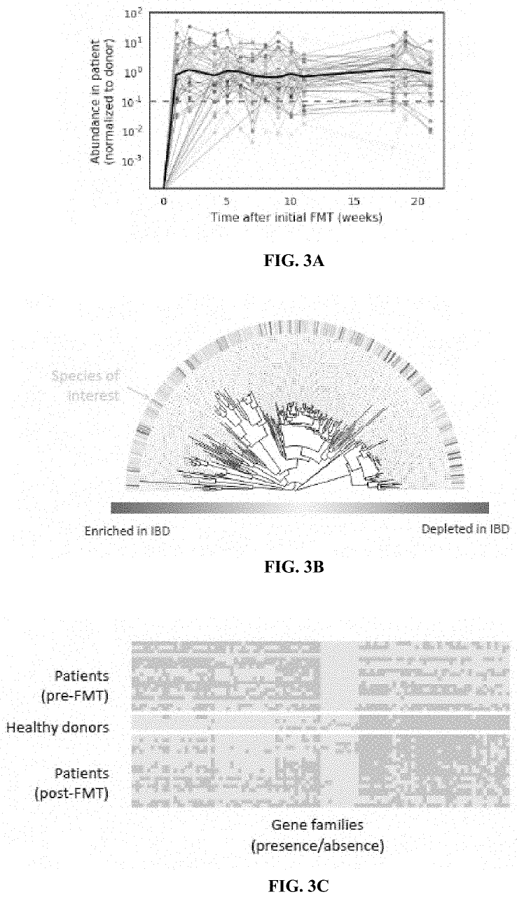

Other conditions, such as inflammatory bowel disease (IBD), involve more subtle changes to patients' microbiomes that cannot be detected using marker-gene abundances alone. For these indications, there is a need for finer-grained analysis of strain-specific genomic features in order to detect engraftment of donor strains at sub-species resolution. FIG. 3A shows that engraftment dynamics after FMT for species missing from the pre-FMT patient but present in the donor can be tracked using marker gene abundances. FIG. 3B shows that, for species present in both pre-treatment patients and in donors, whole-genome data is necessary to observe strain replacement. FIG. 3C shows that a marker gene approach is insufficient for many bacterial species transplanted during FMT, which are present in both pre-FMT and post-FMT patients.

### Example 3: Modeling Engraftment

A method is developed for tracking replacement of recipient strains by donor strains within individual species (FIG. 4A). The strain engraftment model uses a machine-learning model with features measured from mapping shotgun metagenomics data to a database of clustered reference genomes. The 14 features incorporated into the model are: 1) core gene SNP similarity between post-FMT and pre-FMT patient microbiota; 2) core gene SNP similarity between post-FMT patient microbiota and donor microbiota; 3) core gene SNP specificity between post-FMT and pre-FMT patient microbiota (specificity defined as similarity normalized by similarity to unrelated samples); 4) core gene SNP specificity between post-FMT patient microbiota and donor microbiota; 5) gene content similarity between post-FMT and pre-FMT patient microbiota; 6) gene content similarity between post-FMT patient microbiota and donor microbiota; 7) gene content specificity between post-FMT and pre-FMT patient microbiota; 8) gene content specificity between post-FMT patient microbiota and donor microbiota; 9) core gene SNP diversity (heterozygosity) in pre-FMT patient microbiota; 10) core gene SNP diversity (heterozygosity) in post-FMT patient microbiota; 11) core gene SNP diversity (heterozygosity) in donor microbiota; 12) species abundance in pre-FMT patient microbiota; 13) species abundance in post-FMT patient microbiota; and 14) species abundance in donor microbiota. The model does not require assumptions on the number of strains per species, and is not limited to detecting strains with already-sequenced genomes.

Longitudinal datasets are used to construct positive and negative controls to train the model. Controls are constructed using publicly-available metagenomics data of stool samples collected from UC patients and non-IBD controls (HMP2). Positive controls consisted of one sample from a UC patient (representing the patient pre-FMT) and two samples from a control individual taken at different time points (representing the donor and post-FMT patient). Conversely, negative controls consisted of one sample from a control individual (representing the donor) and two longitudinal samples from a UC patient (representing the patient pre- and post-FMT).

The machine learning algorithm is based on Random Forest, as implemented in the publicly-available Python package sklearn. Modifications included an additional null imputation step before model fitting, i.e. inferring missing values for features that couldn't be measured; and changing the default parameter value for the number of trees in the forest. FIG. 4B shows the training set performance averaged 0.82 for all species and 0.96 for abundant species. FIG. 4C shows representative strains showing a false engraftment score (did not engraft in patient) and true engraftment score (engrafted in patient).

### Example 4: Strain Replacement and Patient Outcomes

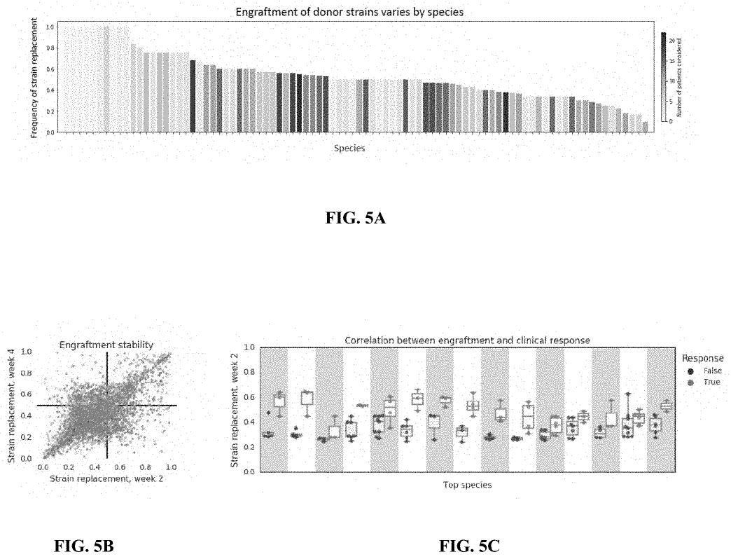

The model from Example 3 is applied to samples from FMT studies in IBD. Using whole-genome sequences of strains isolated from the FMT donors, cases are validated where the algorithm indicates replacement of patient bacterial strains by donor strains. The frequency of strain replacement is calculated for each species, and FIG. 5A verifies that engraftment of donor strains varies by species. FIG. 5B shows that engraftment stability varies by species when tracked across longitudinal samples, here strain replacement at week 2 following FMT and strain replacement at week 4 following FMT. FIG. 5C demonstrates a correlation between engraftment and clinical response. Species where strain replacement was correlated with clinical improvement represent candidates for further exploration of functional drivers of FMT success.

Here we demonstrate that understanding PK is important for learning about how FMT works. PK for FMT and other live biotherapeutics can be defined as engraftment of the strains of the drug.

Measuring changes in species abundances can overlook important strain-level replacement. Here, we describe a machine-learning method to track changes in strain composition in patients receiving FMT. Through the correlation of strain engraftment dynamics with patient outcomes, novel disease mechanisms are discovered.

What is claimed is:

1. A method for treating a disorder related to a gut dysbiosis by increasing an abundance of a bacterial strain in an intestine of a subject, the method comprising:
administering a first dose of a pharmaceutical composition comprising a preparation of uncultured fecal bacteria to the subject, wherein the preparation of uncultured fecal bacteria comprises fecal bacteria from a stool of a healthy human donor, wherein the fecal bacteria comprise the bacterial strain; and
administering a second dose of the pharmaceutical composition to the subject based on a negative engraftment status of the bacterial strain in the intestine of the subject following administration of the first dose of the pharmaceutical composition;
wherein the negative engraftment status is determined by processing via a machine learning model a plurality of DNA sequence-based engraftment metrics, wherein each of the plurality of DNA sequence-based engraftment metrics is representative of an attribute of at least two DNA sequences, wherein the DNA sequences are selected from the group consisting of: (i) a DNA sequence of the fecal bacteria from the stool of the healthy donor; (ii) a DNA sequence of a fecal microbiota from a stool of the subject collected prior to administering the first dose of the pharmaceutical composition; and (iii) a DNA sequence of a fecal microbiota from a stool of the subject collected after administering the first dose of the pharmaceutical composition and before administering the second dose of the pharmaceutical composition.

2. The method of claim 1, wherein each of the plurality of DNA sequence-based engraftment metrics is selected from the group consisting of: core gene SNP similarity between (ii) and (iii); core gene SNP similarity between (i) and (iii); core gene SNP specificity between (ii) and (iii); core gene SNP specificity between (i) and (iii); gene content similarity between (ii) and (iii); gene content similarity between (i) and (iii); gene content specificity between (ii) and (iii); and gene content specificity between (i) and (iii).

3. The method of claim 2, wherein each attribute of each DNA sequence is determined by comparing the DNA sequence to reference bacterial genomes in a database.

4. The method of claim 1, wherein the plurality of DNA sequence-based engraftment metrics further comprise one or more additional engraftment metrics representative of an attribute of only one of (i) to (iii).

5. The method of claim 4, wherein the one or more additional engraftment metrics are selected from the group consisting of: core gene SNP diversity in (i); core gene SNP diversity in (ii); core gene SNP diversity in (iii); species abundance represented in (i); species abundance represented in (ii); and species abundance represented in (iii).

6. The method of claim 1, wherein the machine learning model is trained.

7. The method of claim 6, wherein training of the machine learning model comprises associating the negative engraftment status with one or more attributes of a DNA sequence of a fecal microbiota from an individual having the gut dysbiosis.

8. The method of claim 7, wherein the machine learning model is Random Forest.

9. The method of claim 1, wherein the disorder is inflammatory bowel disease.

10. The method of claim 1, further comprising administering a bacterial isolate to the subject, wherein the bacterial isolate comprises a 16S rRNA sequence that is at least 99% identical to a 16S rRNA sequence of the bacterial strain.

11. The method of claim 10, wherein the second dose of the pharmaceutical composition comprises the bacterial isolate.

12. A method for treating a subject having inflammatory bowel disease, the method comprising:
administering a pharmaceutical composition comprising a preparation of uncultured fecal bacteria to the subject, wherein the preparation of uncultured fecal bacteria comprises fecal bacteria from a stool of a healthy human donor, wherein the fecal bacteria comprise a bacterial strain;
detecting a negative engraftment status of the bacterial strain in the intestine of the subject after administering the pharmaceutical composition, wherein the negative engraftment status is determined by processing via a machine learning model a plurality of DNA sequence-based engraftment metrics, wherein each of the plurality of DNA sequence-based engraftment metrics is representative of an attribute of at least two DNA sequences, wherein the DNA sequences are selected from the group consisting of: (i) a DNA sequence of the fecal bacteria from the stool of the healthy donor; (ii) a DNA sequence of a fecal microbiota from a stool of the subject collected prior to administering the first dose of the pharmaceutical composition; and (iii) a DNA sequence of a fecal microbiota from a stool of the subject collected after administering a first dose of the pharmaceutical composition and before administering a second dose of the pharmaceutical composition; and
administering the bacterial strain to the subject based on detecting the negative engraftment status.

13. The method of claim 12, wherein administering the bacterial strain comprises administering a second dose of the pharmaceutical composition.

14. The method of claim 12, wherein administering the bacterial strain comprises administering to the subject a bacterial mixture comprising cultured bacteria, wherein the cultured bacteria comprise the bacterial strain.

15. The method of claim 14, wherein the bacterial mixture further comprises the preparation of uncultured fecal bacteria.

16. The method of claim 12, wherein each of the plurality of DNA sequence-based engraftment metrics is selected from the group consisting of: core gene SNP similarity between (ii) and (iii); core gene SNP similarity between (i) and (iii); core gene SNP specificity between (ii) and (iii); core gene SNP specificity between (i) and (iii); gene content similarity between (ii) and (iii); gene content similarity between (i) and (iii); gene content specificity between (ii) and (iii); and gene content specificity between (i) and (iii).

17. The method of claim 16, wherein each attribute of each DNA sequence is determined by comparing the DNA sequence to reference bacterial genomes in a database.

18. The method of claim 12, wherein the plurality of DNA sequence-based engraftment metrics further comprise one or more additional engraftment metrics representative of an attribute of only one of (i) to (iii).

19. The method of claim 18, wherein the one or more additional engraftment metrics are selected from the group consisting of: core gene SNP diversity in (i); core gene SNP diversity in (ii); core gene SNP diversity in (iii); species abundance represented in (i); species abundance represented in (ii); and species abundance represented in (iii).

20. The method of claim 12, wherein the machine learning model is trained by associating the negative engraftment status with one or more attributes of a DNA sequence of a fecal microbiota from an individual having inflammatory bowel disease.
